(19) United States
(12) Patent Application Publication
(10) Pub. No.: US 2020/0139116 A1
(43) Pub. Date: May 7, 2020

(54) ELECTRICAL TREATMENT DEVICE, ELECTRONIC DEVICE, AND TERMINAL DEVICE

(71) Applicant: OMRON HEALTHCARE Co., Ltd., Kyoto (JP)

(72) Inventors: Mitsuru SAMEJIMA, Kyoto (JP); Yui WATANABE, Kyoto (JP); Makoto TABATA, Kyoto (JP); Tetsuya SATO, Kyoto (JP); Shozo TAKAMATSU, Kyoto (JP)

(21) Appl. No.: 16/733,311

(22) Filed: Jan. 3, 2020

Related U.S. Application Data
(63) Continuation of application No. PCT/JP2018/022368, filed on Jun. 12, 2018.

(30) Foreign Application Priority Data
Jul. 10, 2017 (JP) 2017-134381

(51) Int. Cl. A61N 1/36

(52) U.S. Cl. CPC A61N 1/36003; A61N 1/0452; A61N 1/36034; A61N 1/36031

(57) ABSTRACT

An electrical treatment device (200) includes an input unit (402) that receives an input of body information relating to a body of a user; a load calculation unit (406) that calculates a load on a knee region of the user on the basis of sensor information and the body information, the sensor information being detected by one or more sensors attached to the knee region of the user; a walking performance calculation unit (408) that calculates a walking performance of the user on the basis of at least acceleration information detected by an acceleration sensor, the acceleration sensor being one of the one or more sensors; and a treatment control unit (410) that controls a voltage applied to a plurality of electrodes that come into contact with the knee region of the user to perform treatment on the knee region. The treatment control unit (410) performs treatment on the knee region at an electrical stimulation intensity appropriate for the user on the basis of the load and the walking performance.

### CROSS-REFERENCE TO RELATED APPLICATIONS

This is a continuation of International Application PCT/JP2018/022368, with an international filing date of Jun. 12, 2018 and JP Application 2017-134381 with a filing date of Jul. 10, 2017, filed by applicant, the disclosure of which is hereby incorporated by reference in its entirety.

### TECHNICAL FIELD

The present disclosure relates to an electrical treatment device, an electronic device, and a terminal device.

### BACKGROUND ART

Electrical stimulation devices that provide electrical stimulation to muscles are known. An example includes an electrical stimulation device described in JP 2012-11102 A (Patent Document 1). This electrical stimulation device includes an attachment portion that attaches to a site on a human body including a joint-spanning muscle, i.e., a muscle that spans a joint, and an electrode portion provided on the attachment portion to deliver a current to the joint-spanning muscle.

### CITATION LIST

### Patent Literature

Patent Document 1: JP 2012-11102 A

### SUMMARY OF INVENTION

### Technical Problem

The electrical stimulation device described in Patent Document 1 is designed to both restrict joint movement in place and minimize or prevent a decrease in muscle power. When a user is determined to be performing an action with a knee-joint load such as walking, a current is delivered to the joint-spanning muscle.

Here, for example, as the walking speed of a lightly fatigued user B can be assumed to be faster than a heavily fatigued user A, the walking performance (for example, walking speed) differs depending on the current state of the user. Thus, even though the load on the knee joint of the user A and that on the user B are the same, the appropriate way of supplying current to a user can be considered to be different between the user A and the user B. In Patent Document 1, current is supplied without taking into account the walking performance of the user.

An object of an aspect of the present disclosure is to provide an electrical treatment device that can perform electrical treatment appropriate for a user when the user is walking. An object of another aspect of the present disclosure is to provide an electronic device that can provide a notification on a walking state appropriate for a user when the user is walking. An object of yet another aspect of the present disclosure is to provide a terminal device that can perform electrical treatment appropriate for a user when the user is walking.

### Solution to Problem

An electrical treatment device according to an embodiment includes an input unit that receives an input of body information relating to a body of a user; a load calculation unit that calculates a load on a knee region of the user on the basis of sensor information and the body information, the sensor information being detected by one or more sensors attached to the knee region of the user; a walking performance calculation unit that calculates a walking performance of the user on the basis of at least acceleration information detected by an acceleration sensor, the acceleration sensor being one of the one or more sensors; and a treatment control unit that controls a voltage applied to a plurality of electrodes that come into contact with the knee region of the user to perform treatment on the knee region. The treatment control unit performs treatment on the knee region at an electrical stimulation intensity appropriate for the user on the basis of the load and the walking performance.

Preferably, the body information includes pain information indicating the amount of pain in the knee region of the user. The walking performance calculation unit calculates a walking speed and a walking speed ratio of the user on the basis of the acceleration information and calculates a walking performance of the user on the basis of the walking speed, the walking speed ratio, and the pain information.

Preferably, when the load is less than a first threshold and the walking performance is equal to or greater than a first performance threshold, the treatment control unit decreases the electrical stimulation intensity from a current intensity to perform treatment on the knee region.

Preferably, when the load is equal to or greater than a first threshold and the walking performance is less than a first performance threshold, the treatment control unit increases the electrical stimulation intensity from a current intensity to perform treatment on the knee region.

Preferably, the body information includes the weight and age of the user. Sensor information further includes angular velocity information detected by an angular velocity sensor, the angular velocity sensor being one of the one or more sensors. The load calculation unit calculates a joint twist of the knee region using the angular velocity information and calculates a load on the knee region of the user on the basis of the weight, the age, and the joint twist of the knee region.

Preferably, a notification unit that provides a notification on a walking state appropriate for the user on the basis of the load and the walking performance is further provided.

Preferably, when the load is less than a first threshold and the walking performance is equal to or greater than a first performance threshold, the notification unit provides a notification on information promoting walking to the user.

Preferably, when the load is equal to or greater than a first threshold and the walking performance is less than a first performance threshold, the notification unit that provides a notification on information promoting easing of walking to the user.

An electronic device according to another embodiment includes an input unit that receives an input of body information relating to a body of a user; a load calculation unit that calculates a load on a knee region of the user on the basis of sensor information and the body information, the sensor information being detected by one or more sensors attached to the knee region of the user; a walking performance calculation unit that calculates a walking performance of the user on the basis of at least acceleration information detected by an acceleration sensor, the acceleration sensor being one of the one or more sensors; and a notification unit that provides a notification on a walking state appropriate for the user on the basis of the load and the walking performance.

A terminal device according to yet another embodiment includes an input unit that receives an input of body information relating to a body of a user; a load calculation unit that calculates a load on a knee region of the user on the basis of sensor information and the body information, the sensor information being detected by one or more sensors attached to the knee region of the user; a walking performance calculation unit that calculates a walking performance of the user on the basis of at least acceleration information detected by an acceleration sensor, the acceleration sensor being one of the one or more sensors; a determination unit that determines an electrical stimulation intensity appropriate for the user on the basis of the load and the walking performance; and an instruction unit that provides an instruction to perform treatment on the knee region in accordance with the determined electrical stimulation intensity to an electrical treatment device that controls a voltage applied to a plurality of electrodes that come into contact with the knee region of the user to perform treatment on the knee region.

### Advantageous Effects of Invention

According to the present disclosure, electrical treatment and a notification appropriate for a user who is walking can be provided.

### BRIEF DESCRIPTION OF DRAWINGS

FIG. 1 is a diagram for describing an overall configuration of a treatment system according to a first embodiment.

FIG. 2 is a block diagram illustrating an example of a hardware configuration of an electrical treatment device according to the first embodiment.

FIG. 3 is a block diagram illustrating a hardware configuration of a sensor device according to the first embodiment.

FIG. 4 is a diagram for describing a treatment method of the electrical treatment device according to the first embodiment.

FIG. 5 is a diagram for describing a notification method of the electrical treatment device according to the first embodiment.

FIG. 6 is a block diagram illustrating a functional configuration of the electrical treatment device according to the first embodiment.

FIG. 7 is a flowchart illustrating an example of a processing procedure of the electrical treatment device according to the first embodiment.

FIG. 8 is a diagram illustrating a schematic configuration of a treatment system according to a second embodiment.

FIG. 9 is a perspective view illustrating a configuration of an electrical treatment device according to the second embodiment.

FIG. 10 is a perspective view illustrating a main body portion, a holder, and a pad of the electrical treatment device according to the second embodiment in a state where the main body portion is separated from the holder and the pad.

FIG. 11 is a block diagram illustrating an example of a hardware configuration of a terminal device according to the second embodiment.

### DESCRIPTION OF EMBODIMENTS

Embodiments of the present invention will be described below with reference to the drawings. In the following description, like components are given like numerals. Names and functions thereof are also the same. Thus, the detailed description of such components is not repeated.

### First Embodiment

### System Configuration

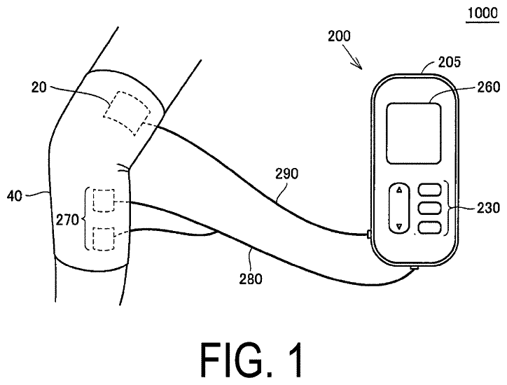

FIG. 1 is a diagram for describing an overall configuration of a treatment system 1000 according to a first embodiment. The treatment system 1000 includes an electrical treatment device 200 and a sensor device 20. The supporter 40 is a knee supporter that entirely covers a knee region of a user. The sensor device 20 is attached to the reverse side of the supporter 40, for example.

Referring to FIG. 1, the electrical treatment device 200 includes, as main constituent elements, a control device 205, which is a main body portion, a pair of pads 270 that attach to a treatment site, a cord 280 for electrically connecting together the control device 205 and the pads 270, and an electrical line 290 for electrically connecting together the control device 205 and the sensor device 20.

The electrical treatment device 200 is a wired type, low-frequency treatment device that performs treatment, such as easing user shoulder stiffness, by supplying a low-frequency pulse current. For example, the frequency of the low-frequency pulse current is from 1 Hz to 1200 Hz. However, the electrical treatment device 200 may be configured to use a pulse current of other frequency bands.

The pads 270 each have a sheet-like shape and are configured to attach to the user's knee region. A first surface of the pad 270 (a surface not in contact with the body) is provided with a plug that corresponds to an electrode (not illustrated) formed on a second surface of the pad 270 (a surface in contact with to the body). The electrode is formed from, for example, a conductive gel-like material or the like. The control device 205 and the pads 270 are connected together by connecting together the plug of the cord 280 and the plug on the pad 270 and inserting the cord 280 into the jack of the control device 205. Note that when the polarity of the electrode formed on one of the pads 270 is positive, the polarity of the electrode formed in the other one of the pads 270 is negative.

The control device 205 is provided with a display 260 and an input interface 230 composed of various buttons. The input interface 230 includes a power button for switching the power on and off, a mode selection button for selecting a treatment mode, a treatment start button, an adjustment button for adjusting the intensity of the electrical stimulation (hereinafter, also referred to as “electrical stimulation intensity”), and the like. Note that the input interface 230 is not limited to the configuration described above, and may be configured to enable various operations by the user as described below. The input interface 230 may be composed of, for example, other buttons, dials, switches, and the like.

The display 260 displays the electrical stimulation intensity, the remaining treatment time, the treatment mode, the attachment state of the pads 270, and the like, and displays various messages.

The control device 205 receives, via the electrical line 290, sensor information detected by the sensor device 20. The control device 205 executes various operations using the sensor information, instruction input information from the user, and the like, performs electrical treatment appropriate for the user, and provides notification on a walking state appropriate for the user.

The sensor device 20 includes various sensors, such as a 3-axis acceleration sensor, and a 3-axis angular velocity sensor. The sensor device 20 outputs sensor information detected by various sensors to the control device 205.

### Hardware Configuration

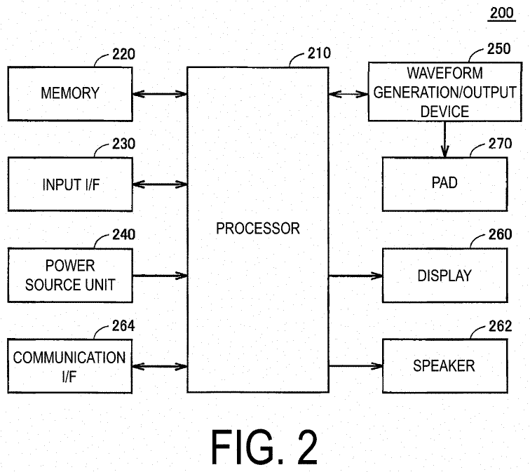

FIG. 2 is a block diagram illustrating an example of a hardware configuration of the electrical treatment device 200 according to the first embodiment. Referring to FIG. 2, the electrical treatment device 200 includes, as main constituent elements, a processor 210, a memory 220, the input interface 230, a power source unit 240, a waveform generation/output device 250, the display 260, a speaker 262, and a communication interface (I/F) 264.

The processor 210 typically includes an arithmetic processing unit, such as a central processing unit (CPU) or a multi processing unit (MPU). The processor 210 functions as a control unit that controls the operation of each component of the electrical treatment device 200 by reading out and executing a program stored in the memory 220. The processor 210 executes processing (steps) of the electrical treatment device 200 described later by executing the program.

The memory 220 is realized by random access memory (RAM), read-only memory (ROM), flash memory, and/or the like. Programs executed by the processor 210, data used by the processor 210, and the like are stored in the memory 220.

The input interface 230 is composed of various buttons such as those described above and receives an operation input for the electrical treatment device 200. When the buttons are operated by the user, signals corresponding to the operations are input to the processor 210.

The power source unit 240 supplies power to the constituent elements of the electrical treatment device 200. As the power source, an alkaline battery may be used, for example. The power source unit 240 stabilizes a battery voltage and generates a drive voltage that is supplied to the constituent elements.

The waveform generation/output device 250 outputs a current (hereinafter, also referred to as “treatment current”) supplied to a treatment site on the body of the user via the pads 270. The waveform generation/output device 250 includes a booster circuit, a voltage adjustment circuit, an output circuit, a current detection circuit, and the like.

The booster circuit boosts the power supply voltage to a predetermined voltage. The voltage adjustment circuit adjusts the voltage boosted by the booster circuit to a voltage corresponding to the electrical stimulation intensity set by the user. Specifically, with the electrical treatment device 200, the electrical stimulation can be adjusted and set to one of a predetermined number of levels (for example, 10 levels) via an adjustment button 238. The processor 210 receives a setting input for the electrical stimulation intensity via the adjustment button 238 and instructs the waveform generation/output device 250 (voltage adjustment circuit) to adjust to a voltage corresponding to the received electrical stimulation intensity.

The output circuit generates a treatment waveform (pulse waveform) corresponding to the treatment mode on the basis of the voltage adjusted by the voltage adjustment circuit and outputs the treatment waveform to (the electrodes of) the pads 270 via the cord 280. Specifically, when an operation, such as switching the treatment mode, changing the electrical stimulation intensity, or the like is performed by the user via the input interface 230, a control signal corresponding to the operation content is input to the output circuit from the processor 210. The output circuit outputs a treatment waveform according to the control signal.

Here, a plurality of treatment modes are prepared in advance in the electrical treatment device 200. Examples of the treatment modes include “massage”, “tap”, “press”, and the like.

The output circuit can generate electrical stimulations corresponding to various modes, such as “massage”, “tap”, “press”, and the like by varying the waveform of the pulses (parameters including pulse width, pulse interval, output polarity). Also, by varying the amplitude of the pulses, the electrical stimulation intensity can be adjusted. For specific treatment waveforms, known waveforms may be utilized. Note that the treatment waveform may be an alternating current waveform rather than a pulse waveform.

The display 260 is composed of a liquid crystal display (LCD), for example, and displays various information in accordance with instructions from the processor 210.

The speaker 262 converts an audio signal from the processor 210 into sound to be output the audio signal to the outside of the electrical treatment device 200. The communication interface 264 is a communication interface for exchanging various data between the control device 205 and the sensor device 20. The communication interface 264 is realized by an adapter, a connector, or the like and is connected to the electrical line 290.

### Sensor Device

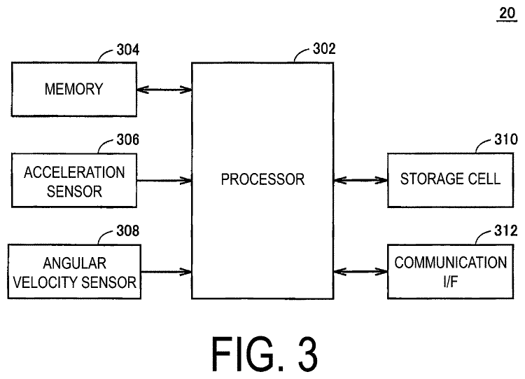

FIG. 3 is a block diagram illustrating a hardware configuration of the sensor device 20 according to the first embodiment. Referring to FIG. 3, the sensor device 20 includes a processor 302 for performing various processes, a memory 304 for storing various information and the like, an acceleration sensor 306, an angular velocity sensor 308, a storage cell 310, and a communication interface (I/F) 312 for communicating with the control device 205.

### Method of Calculating Load

The electrical treatment device 200 according to the present embodiment performs treatment and issues notification appropriate for the user's current condition on the basis of the current load on the user's knee and the current walking performance. First, a method of calculating a knee load will be described.

In the present embodiment, as a result of diligent research by the present inventors, “weight”, “age”, “visual analogue scale (VAS)”, which indicates the amount of knee pain, “knee joint twist”, “degree of leg lift”, and “impact on leg” were selected as factors that affect knee load.

Specifically, the “weight”, “age”, and “VAS” are selected as body information relating to the user's body. The “weight”, “age”, “VAS” are entered, as inputs, by the user via the input interface 230 of the electrical treatment device 200.

VAS is a visual analogue scale and method of objectively evaluating pain or the like, in which the user selects an expression on a line ranging from 0 points to 100 points representing a range from “no pain at all” to “the utmost pain imaginable”. For example, 0 points is defined as “no pain at all” and 100 points is defined as “the utmost pain imaginable”, and the point on the line that the user marks freely is taken as a VAS value.

The “knee joint twist”, “degree of leg lift”, and “impact on leg” are information obtained from acceleration information (acceleration in 3-axis directions) and angular velocity information (angular velocity about the three axes) received from the sensor device 20. For example, the acceleration sensor 306 is provided on the front region of the upper thigh and outputs the acceleration of the upper thigh region rotating about the hip joint during walking. The angular velocity sensor 308 is provided on each of both the upper thigh region and the lower thigh region and outputs the angular velocities of the upper thigh region and the lower thigh region during walking.

The electrical treatment device 200 uses, for example, an output signal (detection result) from the angular velocity sensor 308 to calculate the “knee joint twist” during walking. The “knee joint twist” is estimated, for example, from the difference between the angular velocity of the upper thigh and the angular velocity of the lower thigh. The greater difference, the greater the amount of knee joint twist. Also, the electrical treatment device 200 uses, for example, an output signal from the acceleration sensor 306 to calculate the “degree of leg lift” during walking. The electrical treatment device 200 uses, for example, an output signal from the acceleration sensor 306 to calculate the “impact on leg” during walking. Note that the “knee joint twist”, “degree of leg lift”, and “impact on leg” may be calculated using a known calculation method. A load K on the knee is defined as in Formula 1 below, with variables being “weight”, “age”, “VAS”, “knee joint twist”, “degree of leg lift”, and “impact on leg”.

K=a×“weight”+b×“age”+c×“knee joint twist”+d×“degree of leg lift”+e×“impact on leg”+f×“VAS” (1)

Coefficients a to f are positive constants depending on the respective variables. Thus, the greater the “weight”, “age”, “knee joint twist”, “degree of leg lift”, “impact on leg”, and “VAS”, the greater the load K.

Typically, the load K is calculated for each predetermined control cycle and an average thereof is taken. Specifically, in a case where a predetermined period T is equivalent to n control cycles, the load K of the predetermined period T is the average value of the loads K for n cycles.

### Method of Calculating Walking Performance

As a result of diligent research by the present inventors, “walking speed”, “walking speed ratio”, and “VAS”, which indicates the amount of knee pain, were selected as factors that affect walking performance. The “VAS” is entered as an input by the user via the input interface 230 of the electrical treatment device 200 at certain intervals.

The electrical treatment device 200 uses an output signal from the acceleration sensor 306 to calculate the “walking speed” and the “walking speed ratio”. The “walking speed ratio” is calculated as follows. For example, a current time is defined as ta, a time that is anterior, by a certain interval, to the current time ta (i.e., a time in the past) is defined as tb, a walking speed at the current time ta is defined as V1, and an average walking speed for the time from the time in the past tb to the current time ta is defined as Vav. The walking speed V1 at the current time ta is divided by the average value Vav (=V1/Vav) to obtain the “walking speed ratio” for a certain period. Thus, when a user's “walking speed” decreases, the “walking speed ratio” decreases.

A walking performance W is defined as in Formula 2 below, with the variables being “walking speed”, “walking speed ratio”, and “VAS”.

W=α×“walking speed”+β×“walking speed ratio”+γ×“VAS” (2)

Coefficients α to γ are constants depending on the respective variables. The coefficients α, β are positive constants, and the coefficient γ is a negative constant. Thus, the greater the “walking speed” and the “walking speed ratio” and the smaller the “VAS”, the greater the walking performance W.

Typically, the walking performance W is calculated for each predetermined control cycle and an average thereof is taken. Specifically, the walking performance W for the predetermined period T is an average value of the walking performance W for n cycles.

### Treatment Method

Next, a treatment method of the electrical treatment device 200 will be described. The electrical treatment device 200 provides a treatment method appropriate for the user's condition, on the basis of the load K and the walking performance W calculated as described above. Specifically, the electrical treatment device 200 adjusts the electrical stimulation intensity in accordance with the load K and the walking performance W for the predetermined period T.

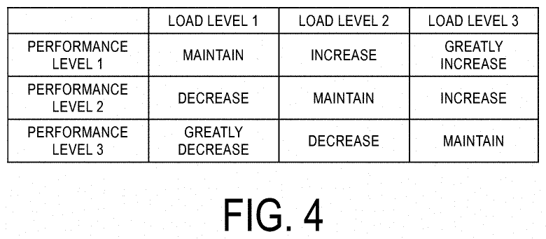

FIG. 4 is a diagram for describing a treatment method of the electrical treatment device 200 according to the first embodiment. Referring to FIG. 4, the load on the user's knee is classified as any of load levels ranging from “load level 1” to “load level 3” depending on the amount of the load K. “Load level 1” means that the load on the knee is low, “load level 2” means that the load is relatively great, and “load level 3” means that the load is great.

For example, when the load K is less than a threshold P1, the load on the user's knee is classified as “load level 1”. When the load K is equal to or greater than the threshold P1 but less than a threshold P2 (which is greater than the threshold P1), the load is classified as “load level 2”, and when the load K is equal to or greater than the threshold P2 but less than a threshold P3 (which is greater than the threshold P2), the load is classified as “load level 3”.

Also, the walking performance of the user is classified as any of performance levels ranging from “performance level 1” to “performance level 3” depending on the magnitude of walking performance W. “Performance level 1” means that the walking performance is low, “performance level 2” means that the walking performance is relatively great, and “performance level 3” means that the walking performance is great.

For example, when the walking performance W is less than a threshold Q1, the walking performance of the user is classified as “performance level 1”. When the walking performance W is equal to or greater than the threshold Q1 but less than a threshold Q2 (which is greater than the threshold Q1), the walking performance is classified as “performance level 2”, and when the walking performance W is equal to or greater than the threshold Q2 but less than a threshold Q3 (which is greater than the threshold Q2), the walking performance is classified as “performance level 3”.

As illustrated in FIG. 4, when the “load level” and “performance level” are the same (for example, “load level 1” and “performance level 1”), the electrical treatment device 200 maintains the current electrical stimulation intensity. This is because, with the load on the knee and the walking performance balanced as such, it is not considered necessary to change the electrical stimulation intensity.

When the “performance level” is one level higher than the “load level” (for example, “load level 1” and “performance level 2”), the electrical treatment device 200 decreases the current electrical stimulation intensity (for example, the electrical stimulation intensity is decreased a predetermined number of levels (two levels, for example)). This is because, with the load on the knee region (taking into account the walking performance) being low relative to the walking performance, it is considered acceptable to decrease the electrical stimulation intensity. This can prevent wasteful power consumption.

When the “performance level” is one level lower than the “load level” (for example, “load level 2” and “performance level 1”), the electrical treatment device 200 increases the current electrical stimulation intensity (for example, the electrical stimulation intensity is raised a predetermined number of levels (two levels, for example)). This is because, with the load on the knee region being great relative to the walking performance, it is considered necessary to increase the electrical stimulation intensity and promote treatment.

When the “performance level” is two levels higher than the “load level” (for example, “load level 1” and “performance level 3”), the electrical treatment device 200 greatly decreases the current electrical stimulation intensity (for example, the electrical stimulation intensity is decreased a predetermined number of levels (four levels, for example)). This is because, with the load on the knee region being very low relative to the walking performance, it is considered acceptable to greatly decrease the electrical stimulation intensity.

When the “performance level” is two levels lower than the “load level” (for example, “load level 3” and “performance level 1”), the electrical treatment device 200 greatly increases the current electrical stimulation intensity (for example, the electrical stimulation intensity is raised a predetermined number of levels (four levels, for example)). This is because, with the load on the knee region being very great relative to the walking performance, it is considered necessary to greatly increase the electrical stimulation intensity and greatly promote treatment.

As described above, the electrical treatment is performed appropriately for the current condition of the user.

### Notification Method

Next, a notification method of the electrical treatment device 200 will be described. The electrical treatment device 200 provides a notification method appropriate for the user's condition, on the basis of the load K and the walking performance W calculated as described above. Specifically, the electrical treatment device 200 provides a notification on a walking state appropriate for the user in accordance with the load K and the walking performance W for the predetermined period T.

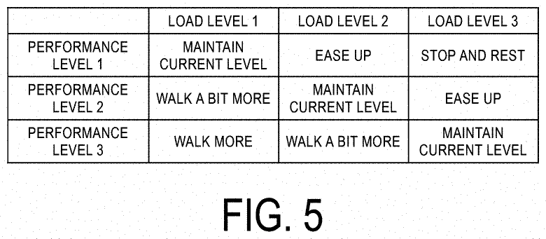

FIG. 5 is a diagram for describing a notification method of the electrical treatment device 200 according to the first embodiment. The “load level” and the “performance level” in FIG. 5 are the same as those in FIG. 4, and thus detailed descriptions thereof will not be repeated.

Referring to FIG. 5, when the “load level” and the “performance level” are the same, the electrical treatment device 200 issues a notification promoting maintaining the current walking state (for example, “maintain current level”). This is because, with the load on the knee and the walking performance balanced as such, it is not necessary to change the current walking state.

When the “performance level” is one level higher than the “load level”, the electrical treatment device 200 issues a notification promoting walking (for example, “walk a bit more”). This is because, with the load on the knee region being low relative to the walking performance, it is considered acceptable to issue a notification to further promote walking.

When the “performance level” is one level lower than the “load level”, the electrical treatment device 200 issues a notification promoting an ease in walking (for example, “ease up”). This is because, with the load on the knee region being great relative to the walking performance, it is considered desirable to issue a notification to slightly ease walking.

When the “performance level” is two levels higher than the “load level”, the electrical treatment device 200 issues a notification promoting walking more (for example, “walk more”). This is because, with the load on the knee region being very low relative to the walking performance, it is considered acceptable to issue a notification to promote walking more.

When the “performance level” is two levels lower than the “load level”, the electrical treatment device 200 issues a notification promoting stop walking (for example, “stop and rest”). This is because, with the load on the knee region being very great relative to the walking performance, it is considered desirable to issue a notification to stop walking talking into account the load on the user's body.

As described above, the notification issued is appropriately for the current condition of the user.

### Functional Configuration

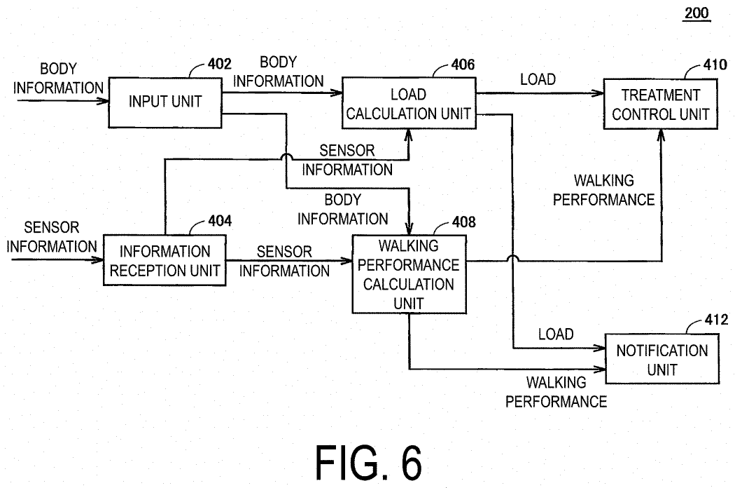

FIG. 6 is a block diagram illustrating a functional configuration of the electrical treatment device 200 according to the first embodiment. Referring to FIG. 6, the electrical treatment unit 200 includes, as main constituent elements, an input unit 402, an information reception unit 404, a load calculation unit 406, a walking performance calculation unit 408, a treatment control unit 410, and a notification unit 412. Each of these functions is realized, for example, by the processor 210 of the electrical treatment device 200 executing a program stored in the memory 220. Note that one or more or all of these functions may be configured to be realized by hardware.

The input unit 402 receives input of body information relating to the user's body. The body information includes user weight, age, and pain information (for example, a “VAS” value) indicating the amount of pain in the knee region.

The information reception unit 404 receives sensor information detected by one or more sensors (for example, the acceleration sensor 306 and the angular velocity sensor 308) attached to the knee region of the user. The sensor information includes acceleration information (an output signal from the acceleration sensor 306) and angular velocity information (an output signal from the angular velocity sensor 308).

The load calculation unit 406 calculates the load on the user's knee on the basis of the sensor information and the body information. Specifically, the load calculation unit 406 calculates the load K in accordance with the “method of calculating load” described above. Note that the load calculation unit 406 may not have to calculate the load K using all of the above-described factors. For example, the load calculation unit 406 may calculate the load K on the knee region of the user on the basis of the knee region joint twist calculated using weight, age, and angular velocity information.

The walking performance calculation unit 408 calculates the walking performance of the user on the basis of the acceleration information and the pain information. Specifically, the walking performance calculation unit 408 calculates the walking performance W in accordance with the “method of calculating walking performance” described above. Note that the walking performance calculation unit 408 may not have to calculate the walking performance W using all of the above-described factors. For example, the walking performance calculation unit 408 may calculate the walking performance W of the user on the basis of the walking speed and the walking speed ratio calculated using the acceleration information.

The treatment control unit 410 performs treatment on the knee region by controlling the voltage applied to the electrodes that come into contact with the knee region of the user. Specifically, the treatment control unit 410 performs treatment on the knee region at an electrical stimulation intensity appropriate for the user on the basis of the load K and the walking performance W. Specifically, the treatment control unit 410 performs treatment in accordance with the “treatment method” described above.

The treatment control unit 410 decreases the electrical stimulation intensity from the current intensity to perform treatment on the knee region when, for example, the load K is less than the threshold P1 (i.e., “load level 1”) and the walking performance W is equal to or greater than the threshold Q1 (i.e., “performance level 2” or “performance level 3”). Also, the treatment control unit 410 increases the electrical stimulation intensity from the current intensity and performs treatment on the knee region when, for example, the load K is equal to or greater than the threshold P1 (i.e., “load level 2” or “load level 3”) and the walking performance W is less than the threshold Q1 (i.e., “performance level 1”).

The notification unit 412 provides a notification on a walking state appropriate for the user on the basis of the load K and the walking performance W. Specifically, the notification unit 412 issues a notification in accordance with the “notification method” described above.

The notification unit 412 provides a notification on information that promotes walking to the user when, for example, the load K is less than the threshold P1 and the walking performance W is equal to or greater than the threshold Q1. Also, the notification unit 412 provides a notification on information saying for the user to ease up walking when, for example, the load K is equal to or greater than the threshold P1 and the walking performance W is less than the threshold Q1. The notification unit 412 may display this information on the display 260 or may output sound on the information via the speaker 262.

### Processing Procedure

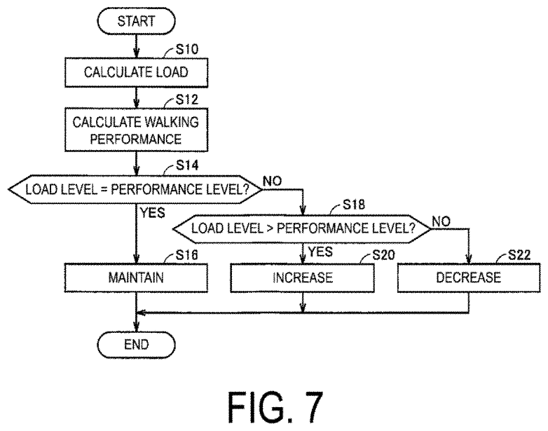

FIG. 7 is a flowchart illustrating an example of a processing procedure of the electrical treatment device 200 according to the first embodiment. Note that in this case, at the start of the processing, the user has attached the pads 270 and the sensor device 20 to the knee region and is walking while wearing the supporter 40, as illustrated in FIG. 1. Also, the electrical treatment device 200 continuously receives sensor information from the sensor device 20, and moreover body information has been input by the user. Also, the electrical stimulation intensity has been set to a predetermined intensity. Typically, each step in FIG. 7 is performed by the processor 210 of the electrical treatment device 200.

Referring to FIG. 7, the processor 210 calculates the load K on the basis of the body information and the sensor information (step S10). Specifically, the processor 210 averages the loads K from the current time to a time that is anterior to the current time by the predetermined period T and calculates the load K for the predetermined period T. The processor 210 calculates the walking performance W on the basis of the acceleration information and the pain information (step S12). The processor 210 calculates the walking performance W for the predetermined period T.

The processor 210 determines whether the load level corresponding to the load K matches the performance level corresponding to the walking performance W (step S14). If they match (YES in step S14), the processor 210 maintains the current electrical stimulation intensity (step S16) and ends the processing. Note that the processor 210 may issue a notification saying to maintain the current walking state.

If they do not match (NO in step S14), the processor 210 determines whether the load level is higher than the performance level (step S18). If the load level is higher than the performance level (YES in step S18), the processor 210 increases the electrical stimulation intensity (step S20) and ends the processing. Note that the processor 210 may issue a notification promoting an ease in walking.

If the load level is lower than the performance level (NO in step S18), the processor 210 decreases the electrical stimulation intensity (step S22) and ends the processing. Note that the processor 210 may issue a notification further promoting walking.

### Advantages

According to the first embodiment, the user can receive electrical stimulation appropriate for the user's body condition. Also, appropriate notifications relating to walking are issued to the user, and this reduces the possibility of the user hurting the knee without being aware of the same.

### Second Embodiment

### System Configuration

A second embodiment described below has a configuration in which the terminal device, the electrical treatment device, and the sensor device are wirelessly connected together and the electrical treatment device performs treatment according to an instruction from the terminal device.

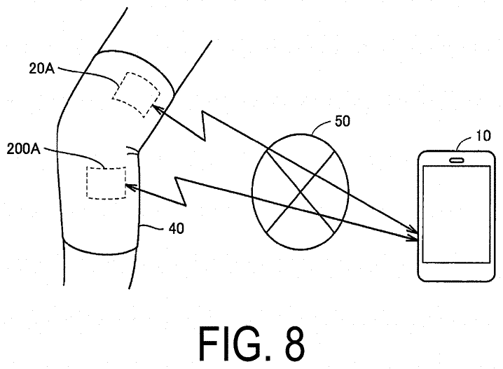

FIG. 8 is a diagram illustrating a schematic configuration of a treatment system 1100 according to the second embodiment. Referring to FIG. 8, the treatment system 1100 includes a terminal device 10, which is a user terminal, an electrical treatment device 200A, a sensor device 20A, and a network 50.

The electrical treatment device 200A is a cordless type and includes a pad, a holder, and a main body portion that are used as a single unit. These portions are used in combination to perform treatment. The specific configuration of the electrical treatment device 200A will be described later.

The terminal device 10 is, for example, a smart phone including a touch panel. In the description hereinafter, a smartphone will be used as a representative example of the “terminal device”. However, the terminal device may be a different terminal device such as a folding type mobile telephone, a tablet terminal device, a personal computer (PC), a personal data assistance (PDA), and the like.

The network 50 for connecting together the terminal device 10, the electrical treatment device 200A, and the sensor device 20A employs a short-range wireless communication system, typically Bluetooth (trademark) low energy (BLE). As such, the terminal device 10, the electrical treatment device 200A, and the sensor device 20A are BLE devices having a function of performing wireless communication using BLE. However, the network 50 is not limited thereto, and a different wireless communication system, such as Bluetooth (trademark) or a wireless local area network (LAN), may be employed.

In the treatment system 1100, the terminal device 10 gives instructions to the paired electrical treatment device 200A via an application installed on the terminal device 10. The terminal device 10 displays various kinds of information on a display 158 of the terminal device 10 and notifies the user of necessary information. For example, the terminal device 10 may display information received from the electrical treatment device 200A on the display 158.

### Configuration of Electrical Treatment Device 200A

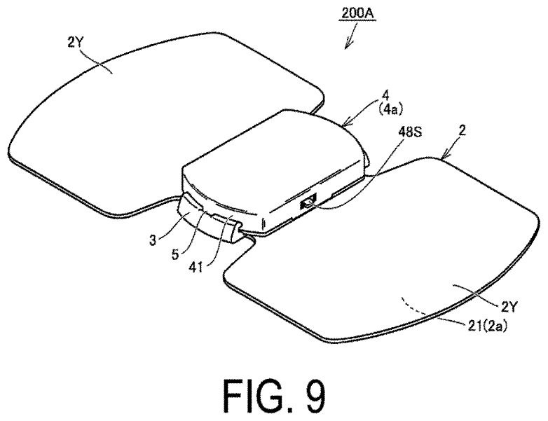

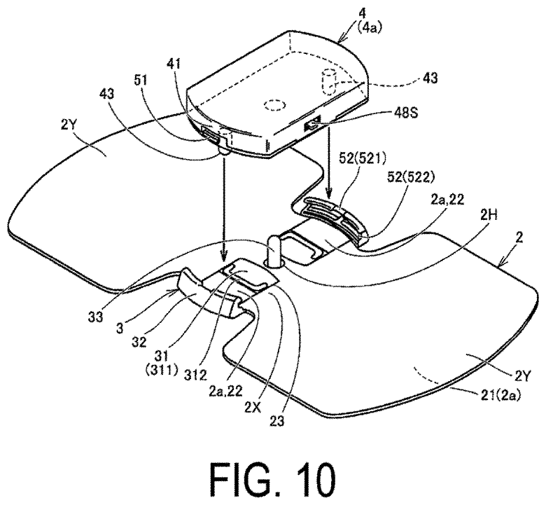

FIG. 9 is a perspective view illustrating a configuration of the electrical treatment device 200A according to the second embodiment. FIG. 10 is a perspective view illustrating a main body portion 4, a holder 3, and a pad 2 of the electrical treatment device 200A according to the second embodiment in a state where the main body portion 4 is separated from the holder 3 and the pad 2.

Referring to FIGS. 9 and 10, the electrical treatment device 200A is a so-called cordless type low-frequency treatment device and includes the pad 2, the holder 3, and the main body portion 4.

The pad 2 has a sheet-like shape and is configured to attach to the user's body. A conductive layer 2a is provided on a body-side portion 21 surface (lower surface), of the outer surfaces of the pad 2, that faces the body. The pad 2 is attached to the user's skin by using a conductive gel or the like, and a low-frequency pulse current is supplied to the user through the conductive layer 2a.

Referring to FIG. 9, the pad 2 includes an attachment portion 2X and a treatment portion 2Y. The attachment portion 2X is held by the holder 3. A window portion 23 and a through hole 2H are provided at the attachment portion 2X. A positioning protrusion 312 of the holder 3 is disposed on the inside of the window portion 23. An interlock pin 33 of the holder 3 is inserted through the through hole 2H. The treatment portion 2Y is provided on both the left and right sides of the attachment portion 2X, and the conductive layer 2a is exposed on the body-side portion 21 of the treatment portion 2Y.

The conductive layer 2a is also exposed on the surface facing the main body portion 4 at the attachment portion 2X, and the exposed portion constitutes a pad side electrode portion 22. The pad side electrode portion 22 is formed to establish an electrical connection with a main body portion side electrode portion 43, and a conductive layer 2a corresponding to one electrode portion (for example, a positive electrode) is exposed at one end of the attachment portion 2X, and a conductive layer 2a corresponding to another electrode portion (for example, a negative electrode) is exposed at the other end of the attachment portion 2X.

Referring to FIG. 10, the holder 3 includes a pad holding portion 31 with a plate-like shape and a pair of wall portions 32 erected from both ends of the pad holding portion 31. An attachment portion 2X of the pad 2 is disposed on an upper surface 311 of the pad holding portion 31. Double-sided adhesive tape, glue, adhesive, or the like is disposed, as necessary, between the upper surface 311 and the attachment portion 2X.

The positioning protrusion 312 is provided on the pad holding portion 31. By fitting the inner peripheral edge of the window portion 23 provided in the pad 2 to the positioning protrusion 312, the pad 2 can be positioned with respect to the holder 3. The interlock pin 33 is centrally disposed on the pad holding portion 31. When attaching the pad 2 to the holder 3, the interlock pin 33 is inserted into the through hole 2H.

The pad 2 is a consumable item, and the pad 2 can be detachably attached to the main body portion 4, thus allowing replacement of the pad 2. In the present embodiment, the holder 3 holds the pad 2 such that the holder 3 and the pad 2 are integrated, and the main body portion 4 is configured to be detachably attached to the pad 2 and the holder 3. The pad 2 can be replaced together with the holder 3, or it is also possible to reuse the holder 3 as necessary.

Referring to FIGS. 9 and 10, the main body portion 4 includes a case 4a with a substantially rectangular parallelepiped shape as an outer cover. A guiding/engagement portion 5 (FIG. 9) is formed between the case 4a and the holder 3, and the main body portion 4 (case 4a) is detachably attached to the holder 3. The guiding/engagement portion 5 includes a protrusion 51 (FIG. 10) formed on a side surface 41 of the case 4a and a groove portion 52 (FIG. 10) formed in each of the wall portions 32 of the holder 3.

Referring to FIG. 9, the groove portion 52 includes a vertical groove portion 521 and a lateral groove portion 522. The vertical groove portion 521 is formed in the vertical direction and opens upward. The lateral groove portion 522 is formed in the lateral direction and opens at both ends. When the main body portion 4 is attached to the holder 3, both of the protrusion 51 and the groove portion 52 move closer together in a facing direction and engage together. By rotating and moving the main body portion 4 with respect to the holder 3, the engagement between the two is released, and the main body portion 4 can be removed from the holder 3.

With the main body portion 4 attached to the holder 3, the main body portion 4 supplies a low-frequency pulse current to the conductive layer 2a of the pad 2. Specifically, the main body portion 4 includes a pair of the main body portion side electrode portions 43, a substrate (not illustrated), an electric circuit (not illustrated), and an interlock mechanism (not illustrated). The electric circuit includes various control devices and is mounted on the surface of the substrate.

The control devices may include a processor for performing various processes, a memory for storing programs, data, and the like, a communication interface for wirelessly communicating various types of data with the terminal device 10, and a waveform generation/output device and the like for boosting the power source voltage, generating and outputting a low-frequency pulse current (treatment current).

The substrate, the electric circuit, and the interlock mechanism are provided inside the main body portion 4 (case 4a). A power source (not illustrated) such as a battery is also provided inside the main body portion 4 (case 4a). A display portion (not illustrated) such as a switch 48s, light emitting diode (LED), and a button (not illustrated) are provided on the outside of the case 4a.

In a state in which the main body portion 4 is attached to the holder 3, an end portion of the main body portion side electrode portion 43 abuts the pad side electrode portion 22. Thus, the main body portion side electrode portion 43 and the pad side electrode portion 22 are electrically connected, whereby the electric circuit can supply a low-frequency pulse current to the pad side electrode portion 22.

### Configuration of Terminal Device 10

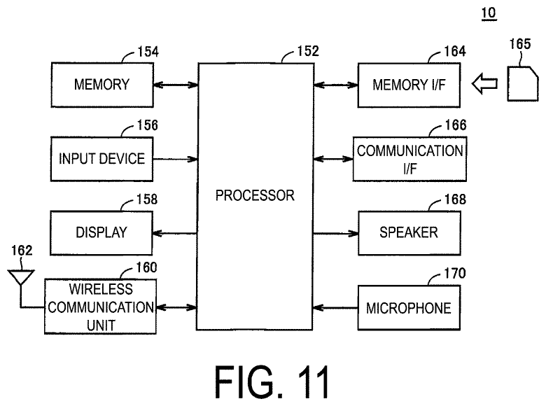

FIG. 11 is a block diagram illustrating an example of a hardware configuration of the terminal device 10 according to the second embodiment. Referring to FIG. 11, the terminal device 10 includes, as main components, a processor 152, a memory 154, an input device 156, a display 158, a wireless communication unit 160, a memory interface (I/F) 164, a communication interface (I/F) 166, a speaker 168, and a microphone 170.

The processor 152 typically may be an arithmetic processing unit such as a central processing unit (CPU) or a multi processing unit (MPU). The memory 154 is realized by random access memory (RAM), read-only memory (ROM), flash memory, and the like.

The input device 156 receives an operation input to the terminal device 10. Typically, the input device 156 is realized by a touch panel. The touch panel is provided on the display 158 that functions as a display portion, and is, for example, an electrostatic capacitive type. The touch panel detects touch operations on the touch panel by an external object at predetermined intervals of time and inputs touch coordinates to the processor 152. However, the input device 156 may be a button or the like.

The wireless communication unit 160 connects to a mobile communication network via a communication antenna 162 and transmits and receives signals for wireless communication. Accordingly, the terminal device 10 can communicate with other communication devices via a mobile communication network such as long term evolution (LTE), for example.

The memory interface 164 reads data from an external storage medium 165. The processor 152 reads the data stored in the storage medium 165 via the memory interface 164 and stores the data in the memory 154. The processor 152 reads the data from the memory 154 and stores the data in the external storage medium 165 via the memory interface 164.

The storage medium 165 may also be a compact disc (CD), digital versatile disk (DVD), Blu-ray (trademark) disc (BD), universal serial bus (USB) memory, secure digital (SD) memory card, and the like that store programs in a non-volatile manner.

The communication interface (I/F) 166 is a communication interface for exchanging various data between the electrical treatment device 200A and the sensor device 20A and is realized by an adapter, a connector, or the like. As the communication method, for example, a wireless communication method such as Bluetooth (trademark) low energy (BLE), wireless LAN, and the like may be employed.

The speaker 168 converts an audio signal from the processor 152 to voice and outputs the same to the outside of the terminal device 10. The microphone 170 receives an audio input for the terminal device 10 and provides to the processor 152 an audio signal corresponding to the audio input.

### Functional Configuration

The terminal device 10 according to the second embodiment has the functions corresponding to those of the input unit 402, the information reception unit 404, the load calculation unit 406, the walking performance calculation unit 408, and the notification unit 412, among configuring functions of the electrical treatment device 200 illustrated in FIG. 6.

The terminal device 10 further includes a determination unit that determines an electrical stimulation intensity appropriate for the user on the basis of the load K and the walking performance W. The method of determining the electrical stimulation intensity is similar to that in the “treatment method” described above. For example, when the “load level” and “performance level” are the same, the determination unit determines to maintain the current electrical stimulation intensity. When the “performance level” is higher than the “load level”, the determination unit determines to decrease the electric stimulation intensity. When the “performance level” is lower than the “load level”, the determination unit determines to increase the electric stimulation intensity.

The terminal device 10 further includes an instruction unit that provides an instruction to the electrical treatment device 200A to perform the treatment on the knee region according to the determined electrical stimulation intensity. The electrical treatment device 200A performs treatment on the knee region at an electrical stimulation intensity in accordance with an instruction from the instruction unit.

### Advantages

According to the second embodiment, advantages similar to those of the first embodiment can be obtained.

### OTHER EMBODIMENTS

(1) In the first embodiment described above, a configuration has been described in which the electrical treatment device performs electrical treatment and issues a notification appropriate for the user on the basis of the load K and the walking performance W, but no such limitation is intended. The electrical treatment device may not perform treatment and may provide a notification on a walking state appropriate for the user in accordance with the load K and the walking performance W. In this case, the electronic device has all functional configurations of the electrical treatment device 200 in FIG. 6 except that of the treatment control unit 410.

(2) In the first embodiment described above, a configuration has been described in which the electrical treatment device 200 and the sensor device 20 are separate devices. However, no such limitation is intended, and the electrical treatment device 200 may include all of the sensors. In this case, for example, the electrical treatment device 200 is directly electrically connected to each sensor via an electrical line or the like.

(3) In the embodiments described above, the terms “weight”, “age”, “VAS”, “knee joint twist”, “degree of leg lift”, and “impact on leg” are selected as factors affecting the knee, but factors are not limited to these factors. For example, the load K may be calculated by further taking into account “walking time”, “center of gravity”, “degree of leg bend”, “body temperature”, and “fatigue”.

The “walking time” is measured as the elapsed time from the start time of walking by using a timing (timer) function included in the electrical treatment device 200. The start time of walking is, for example, a point in time when the user presses a walking start button via the input interface 230. The “center of gravity” is calculated as a ratio between a pressure value from a pressure sensor attached to the sole of one foot and a pressure value from a pressure sensor attached to the sole of the other foot. The “degree of leg bend” is a value detected by a bending sensing sensor attached to the back of the knee. The “body temperature” is detected by a temperature sensor attached at any location on the user's body. The “fatigue” is the level of blood oxygen saturation detected by a pulse oximeter. In this case, the electrical treatment device 200 is electrically connected to or configured to be able to communicate with each of the pressure sensors, the bend sensing sensor, the temperature sensor, and the pulse oximeter.

The load K is defined as in Formula (3) below.

K=a×“weight”+b×“age”+c×“knee joint twist”+d×“degree of leg lift”+e×“impact on leg”+f×“VAS”+g×“walking time”+h×“center of gravity”+i×“degree of leg bend”+j×“body temperature”+k×“fatigue” (3)

Coefficients a to k are constants depending on respective variables. Coefficients a to j are positive constants, and coefficient k is a negative constant.

(4) In the embodiments described above, a program may be provided that causes a computer to function and execute controls such as those described in the flowcharts described above. Such a program can also be provided as a program product stored on a non-temporary computer-readable recording medium, such as a flexible disk, a compact disk read only memory (CD), a secondary storage device, a main storage device, a memory card, and the like attached to a computer. Alternatively, a program may be provided, which is stored on a recording medium such as a hard disk built into a computer. The program may also be provided by download via a network.

With the program, required modules from among program modules provided as part of the computer operating system (OS) may be called in a predetermined sequence at a predetermined timing to execute processing. In this case, the modules described above are not included in the program itself, and the process is executed in cooperation with the OS. Programs that do not include such modules may also be included in the program according to the present embodiment.

In addition, the program according to the present embodiment may be provided integrated into a part of another program. In this case as well, the modules included in the other programs described above are not included in the program itself, and the process is executed in cooperation with the other programs. Programs included in such other programs may also be included in the program according to the present embodiment.

(5) The configuration given as an example of the embodiment described above is an example configuration of the present invention. The configuration can be combined with other known technology, and parts thereof may be omitted or modified within the scope of the present invention. Furthermore, the processes and configurations of other embodiments may be employed as appropriate to the embodiments described above.

The embodiments described herein are illustrative in all respects and are not intended as limitations. The scope of the present invention is indicated not by the descriptions above but by the claims and includes all meaning equivalent to the scope and changes within the scope.

### REFERENCE SIGNS LIST

2, 270 Pad
2H Through hole
2X Attachment portion
2Y Treatment portion
2a Conductive layer
3 Holder
4 Body portion
4a Case
5 Guiding/engagement portion
10 Terminal device
20, 20A Sensor device
21 Body-side portion
22 Pad side electrode portion
23 Window portion
31 Pad holding portion
32 Wall portion
33 Interlock pin
40 Supporter
41 Side surface
43 Main body portion side electrode portion
48s Switch
50 Network
51 Protrusion
52 Groove portion
152, 210, 302 Processor
154, 220, 304 Memory
156 Input device
158, 260 Display
160 Wireless communication unit
162 Communication antenna
164 Memory interface
165 Storage medium
168, 262 Speaker
170 Microphone
200, 200A Electrical treatment device
205 Control device
230 Input interface
238 Adjustment button
240 Power source unit
250 Waveform generation/output device
280 Cord
290 Electrical line
306 Acceleration sensor
308 Angular velocity sensor
310 Storage cell
311 Upper surface
312 Positioning protrusion
402 Input unit
404 Information reception unit
406 Load calculation unit
408 Walking performance calculation unit
410 Treatment control unit
412 Notification unit
521 Vertical groove portion
522 Lateral groove portion
1000, 1100 Treatment system

1. An electrical treatment device, comprising:
an input unit configured to receive an input of body information relating to a body of a user;
a load calculation unit configured to calculate a load on a knee region of the user on the basis of sensor information and the body information, the sensor information being detected by one or more sensors attached to the knee region of the user;
a walking performance calculation unit configured to calculate a walking performance of the user on the basis of at least acceleration information detected by an acceleration sensor, the acceleration sensor being one of the one or more sensors; and
a treatment control unit configured to control a voltage applied to a plurality of electrodes that come into contact with the knee region of the user to perform treatment on the knee region, wherein
the treatment control unit is configured to perform treatment on the knee region at an electrical stimulation intensity appropriate for the user on the basis of the load and the walking performance.

2. The electrical treatment device according to claim 1, wherein
the body information is configured to comprise pain information indicating an amount of pain in the knee region of the user; and
the walking performance calculation unit is configured to calculate a walking speed and a walking speed ratio of the user on the basis of the acceleration information and calculates a walking performance of the user on the basis of the walking speed, the walking speed ratio, and the pain information.

3. The electrical treatment device according to claim 1, wherein when the load is less than a first threshold and the walking performance is equal to or greater than a first performance threshold, the treatment control unit is configured to decrease the electrical stimulation intensity from a current intensity to perform treatment on the knee region.

4. The electrical treatment device according to claim 1, wherein when the load is equal to or greater than a first threshold and the walking performance is less than a first performance threshold, the treatment control unit is configured to increase the electrical stimulation intensity from a current intensity to perform treatment on the knee region.

5. The electrical treatment device according to claim 1, wherein
the body information is configured to comprise a weight and an age of the user;
the sensor information is configured to further comprise angular velocity information detected by an angular velocity sensor, the angular velocity sensor being one of the one or more sensors; and
the load calculation unit is configured to calculate a joint twist of the knee region using the angular velocity information and calculates a load on the knee region of the user on the basis of the weight, the age, and the joint twist of the knee region.

6. The electrical treatment device according to claim 1, further comprising a notification unit configured to provide a notification on a walking state appropriate for the user on the basis of the load and the walking performance.

7. The electrical treatment device according to claim 6, wherein when the load is less than a first threshold and the walking performance is equal to or greater than a first performance threshold, the notification unit is configured to provide a notification on information promoting walking to the user.

8. The electrical treatment device according to claim 6, wherein when the load is equal to or greater than a first threshold and the walking performance is less than a first performance threshold, the notification unit is configured to provide a notification on information promoting easing of walking to the user.

9. An electronic device, comprising:
an input unit configured to receive an input of body information relating to a body of a user;
a load calculation unit configured to calculate a load on a knee region of the user on the basis of sensor information and the body information, the sensor information being detected by one or more sensors attached to the knee region of the user;
a walking performance calculation unit configured to calculate a walking performance of the user on the basis of at least acceleration information detected by an acceleration sensor, the acceleration sensor being one of the one or more sensors; and
a notification unit configured to provide a notification on a walking state appropriate for the user on the basis of the load and the walking performance.

10. A terminal device, comprising:
an input unit configured to receive an input of body information relating to a body of a user;
a load calculation unit configured to calculate a load on a knee region of the user on the basis of sensor information and the body information, the sensor information being detected by one or more sensors attached to the knee region of the user;
a walking performance calculation unit configured to calculate a walking performance of the user on the basis of at least acceleration information detected by an acceleration sensor, the acceleration sensor being one of the one or more sensors;
a determination unit configured to determine an electrical stimulation intensity appropriate for the user on the basis of the load and the walking performance; and
an instruction unit configured to provide an instruction to perform treatment on the knee region in accordance with the determined electrical stimulation intensity to an electrical treatment device that controls a voltage applied to a plurality of electrodes that come into contact with the knee region of the user to perform treatment on the knee region.
